(19) United States
(12) Patent Application Publication
(10) Pub. No.: US 2005/0222651 A1
(43) Pub. Date: Oct. 6, 2005

(54) Therapeutic device and method

(75) Inventor: Herbert C. Jung JR., Spartanburg, SC (US)

Correspondence Address: DORITY & MANNING, P.A., POST OFFICE BOX 1449, GREENVILLE, SC, 29602-1449, US

(73) Assignee: CHEROKEE PRODUCTS, INC

(21) Appl. No.: 11/091,357

(22) Filed: Mar. 28, 2005

Related U.S. Application Data
(63) Continuation-in-part of application No. 10/284,813, filed on Oct. 31, 2002.

(51) Int. Cl. A61F 7/00

(52) U.S. Cl. 607/104; 607/108

(57) ABSTRACT

A therapeutic device and method for providing therapeutic relief are provided. A vapor permeable outer cover is secured to a vapor permeable inner cover. The outer cover and inner cover define at least one enclosed interior space therebetween with both the outer and inner cover being permeable to microwaves. The inner cover defines an inner surface that forms a collapsible appendage receiving chamber. The outer cover and the inner cover form at least one opening leading into the appendage receiving chamber. The at least one opening and the appendage receiving chamber are thereby capable of receiving an appendage of a user. A water absorbent material is disposed within said interior space between the outer and inner covers. The water absorbent material is capable of water vapor release upon application of energy thereto.

### CROSS REFERENCE TO RELATED APPLICATION

The present application is a continuation in part application of U.S. patent application having Ser. No. 10/284,813, which was filed on Oct. 31, 2002.

### BACKGROUND OF THE INVENTION

Many people suffer from sinus headaches, lung congestion, asthma, or related respiratory problems. Various methods and apparatus have been developed to provide relief from symptoms associated with such conditions.

Steam-generating devices, for example, vaporize water into the air for inhalation. Unfortunately, existing steam-generating devices may have significant drawbacks. Steam-generating devices for the home usually are relatively expensive. Furthermore, maintaining a steam-generating device in the home requires frequent maintenance, including periodic re-filling of the device with water. Electrical steam generating apparatus requires costly electrical power to operate. Many steam-generating devices are limited with regard to portability. Such devices typically must produce much more total water vapor than is actually ingested by the patient. It is sometimes necessary to fill an entire room with water vapor before achieving an ambient vapor concentration sufficient to provide desirable effects upon a user.

Some inhalation devices have been designed for placement directly into the mouth, thereby facilitating respiratory treatment by direct inhalation of therapeutic agents. However, direct methods for administering therapeutic agents also have disadvantages. Many such inhalation devices are not adapted for use with moist heat or steam. For many users, however, application of heated water vapor assists in relieving symptoms relating to such illnesses.

U.S. Pat. No. 5,630,961 to Salee is directed to a thermal storage composition activated by exposure to microwave energy. The composition comprises a mixture of at least two impregnated powders wherein a first powder is calcium silicate, and a second powder is calcium silicate impregnated with a phase-change material. Another patent, U.S. Pat. No. 5,366,491 pertains to a microwave-activated moist heat apparatus for the body. U.S. Pat. No. 5,375,278 is directed to a therapeutic pillow having a removable washable outer sleeve. The pillow has a bag-like cover filled with granular material having a water content in the range of 5% to 25% by weight. U.S. Pat. No. 5,584,086 is related to the patent described above (i.e. the U.S. Pat. No. 5,375,278) and further describes a therapeutic pillow having a bag-like cover filled with natural granular material. U.S. Pat. No. 3,848,281 is directed to a foam rubber cushion suitable for use as an infant headrest or the like. U.S. Pat. No. 2,973,762 describes a steam cloth that is useful during facial treatments in a barber shop or a beauty salon.

Many people also suffer from arthritis, myalgia, neuropathy, carpel tunnel syndrome, and other circulatory, neurological, muscle and joint ailments. Such ailments are often particularly painful for the extremities or appendages of the sufferer. It has been found that a moist heat applied to such appendages can be beneficial for relieving such pain. For example, arthritis commonly attacks the joints of appendages such as the hand. When the joints in the hand ache, a hot and wet towel may be applied around those aching joints. By wrapping the hand in the towel, the joint will be surrounded by the therapeutic moist heat. The sufferer will almost immediately receive relief from the pain through this application of this moist heat.

The use of wet hot towels to wrap around the afflicted appendage, however, creates an inconvenience due to the moisture within the towel. It is hard to regulate the amount of moisture contained in the towel. So often, the wet hot towels tend to get the clothes of the sufferer wet. Further, once the heat dissipates or the sufferer has gained her desired relief, the sufferer is left with a wet towel with which to dispense or wash.

Therefore, a need exists for a reusable therapeutic device for efficiently applying moist heat around an appendage of a sufferer that is convenient and inexpensive to use.

### SUMMARY OF THE INVENTION

According to certain aspects of the present subject matter, a therapeutic treatment device provides a vapor permeable outer cover secured to a vapor permeable inner cover. The vapor permeable outer cover and the vapor permeable inner cover define at least one enclosed interior space between the vapor permeable outer cover and the vapor permeable inner cover with both the vapor permeable outer cover and vapor permeable inner cover being permeable to microwaves. The vapor permeable outer cover defines an outer surface, while the vapor permeable inner cover defines an inner surface. The inner surface of the vapor permeable inner cover is configured to define a collapsible appendage receiving chamber therebetween. Further, the vapor permeable inner cover forms at least one opening leading into the appendage receiving chamber. The at least one opening and the appendage receiving chamber is capable of receiving an appendage of a user with the appendage receiving chamber being larger in cross-sectional size than the at least one opening.

A water absorbent material is disposed within the interior space defined between the vapor permeable outer cover and the vapor permeable inner cover. Upon application of energy, this water absorbent material is capable of water vapor release. In this manner, the water absorbent material may release water vapor through the vapor permeable outer cover and the vapor permeable inner cover.

According to another aspect of the present subject matter, a therapeutic treatment device provides a first cushion having a first vapor permeable cover defining an enclosed first interior space and a second cushion having a second vapor permeable cover defining an enclosed second interior space. The first cushion is secured to the second cushion so that a collapsible chamber is defined between the first and second cushions. Further, the first and second cushions form at least one opening leading into the collapsible chamber. A water absorbent material is positioned within the first interior space of the first cushion and the second interior space of the second cushion. The water absorbent material is capable of water uptake at ambient temperatures and water vapor release upon application of energy to the water absorbent material. The first and second cushions drape around an appendage of a user when the appendage is disposed in the collapsible chamber so as to effective surround the appendage.

According to a further aspect of the present subject matter, a method for producing therapeutic relief in an appendage of a user of a therapeutic device is provided. A vapor permeable outer cover secured to a vapor permeable inner cover is provided so that an interior space between the outer cover and inner cover is defined. The inner cover is configured to define a collapsible appendage receiving chamber and an opening with the appendage receiving chamber being larger in cross-sectional size than the opening. Further, a water absorbent material is provided within the interior space between the outer cover and the inner cover.

The outer cover, the inner cover the water absorbent material are heated by having energy applied thereto. A user may then insert an appendage through the opening defined by the inner cover into the chamber. Heat is radiated and water vapor is released from the water absorbent material to treat the appendage disposed within the chamber of the inner cover.

The features of the subject matter will be described in greater detail through the use of the appended figures.

### BRIEF DESCRIPTION OF THE DRAWINGS

A full and enabling disclosure of this invention, including the best mode shown to one of ordinary skill in the art, is set forth in the specification. The following Figures illustrate the invention:

FIG. 1 is a perspective view of an exemplary present vapor therapy treatment device including a pouch attached to a larger cushion;

FIG. 2 shows a further perspective view of an exemplary present vapor therapy treatment device in which the pouch is disengaged from the cushion;

FIG. 3 illustrates one step in the method of the invention whereby an exemplary cushion having a water absorbing material therein is placed within a microwave oven and subjected to microwaves for a period of time, in accordance with present subject matter;

FIG. 4 displays one method of application of a vapor therapy material or composition to a vapor permeable pouch;

FIG. 5 shows a cross-section taken along line 5-5 of FIG. 1 revealing the transmission of heated water vapor through the vapor permeable cover and into the pouch, causing release of therapeutic vapors from the pouch;

FIG. 6 is a schematic diagram showing one method of producing a therapeutic vapor for application to the facial area of a user;

FIG. 7 is a perspective view of a therapeutic device according to the present subject matter into which a user may insert an appendage;

FIG. 8 is a top view a therapeutic device as shown in FIG. 7;

FIG. 9 is a perspective view of a therapeutic as shown in FIG. 7 being used to provide relief to a hand;

FIG. 10 is a perspective view of a therapeutic as shown in FIG. 7 being used to provide relief to a foot;

FIG. 11 is a cross-sectional view taken along line 11-11 of FIG. 7 of the therapeutic device; and

FIG. 12 is a schematic diagram showing one method of preparing a therapeutic device for providing relief to of a user's appendage.

### DETAILED DESCRIPTION OF THE INVENTION

Reference now will be made to the embodiments of the invention, one or more examples of which are set forth below. Each example is provided by way of explanation of the invention, not as a limitation of the invention. In fact, it will be apparent to those skilled in the art that various modifications and variations can be made in this invention without departing from the scope or spirit of the invention.

A vapor therapy treatment device includes a cushion 20 of cotton flannel material forming vapor permeable cover 21. A vapor permeable pouch 23 having an opening 30 is releasably attached to the outer surface 22 of the vapor permeable cover 21.

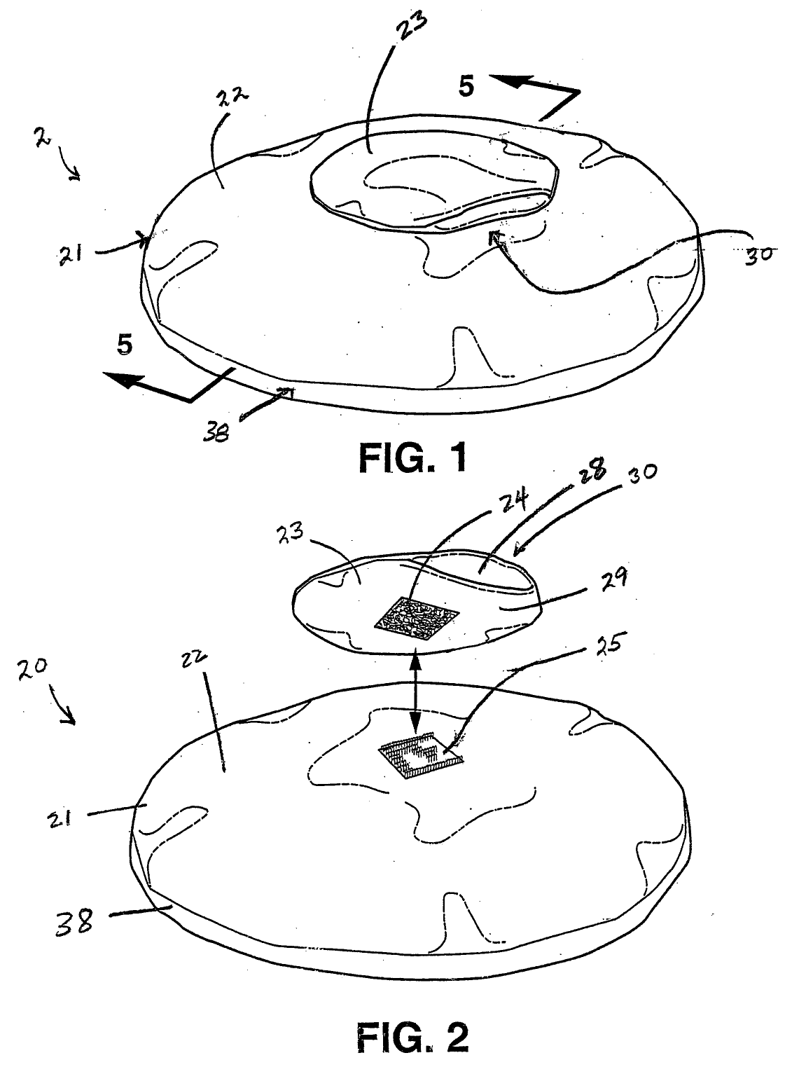

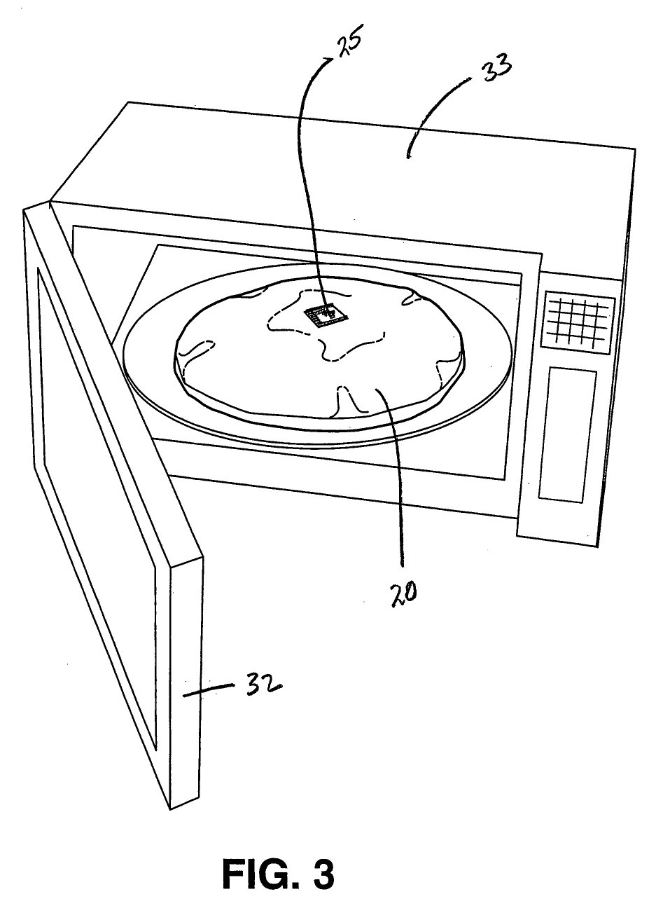

In the embodiment of the invention shown in FIGS. 1-2, the vapor permeable pouch 23 is selectively detachable from the outer surface 22 of the vapor permeable cover 21. FIG. 2 employs a fastener which includes a first component 24 and a second component 25 which mate to each other. In the particular embodiment of FIG. 2, the respective first and second components 24-25 comprise a hook and loop type fastening system. The hook portion of the fastener is the second component 25, while the loop portion of the fastener is the first component 24. In other applications of the invention, however, the loop portion of the fastener could be provided as second component 25 upon the outer surface 22 of the vapor permeable cover 21. In such an embodiment, the hook portion of the fastener could reside upon the vapor permeable pouch 23. Thus, in either embodiment, it is possible to disengage and re-attach the pouch 23 to the vapor permeable cover 21, as needed.

Other embodiments of the invention may employ other types of fasteners. For example, snap-type fasteners, buttons, zippers, or other mechanical means can be used to releaseably engage the vapor permeable pouch 23 to the vapor permeable cover 21. The invention is not limited to any particular means of engaging or disengaging the vapor permeable pouch 23 to the vapor permeable cover 21.

The cushion 20 includes vapor permeable cover 21 enclosing water absorbing material 41 (see cross-sectional view along line 5-5 in FIG. 5). In the use of the invention, the water absorbing material 41 is capable of uptake or adsorption of water at ambient temperatures. Thus, water is retained within the water absorbing material 41 at ambient temperatures. In FIG. 5, the top panel 48 and bottom panel 49 are joined by stitching at seam 38 to form the vapor permeable cover 21. The vapor permeable cover 21 encloses water absorbing material 41 which upon being heated by an energy source, such as microwave energy, causes water vapor 39, to permeate through top panel 48.

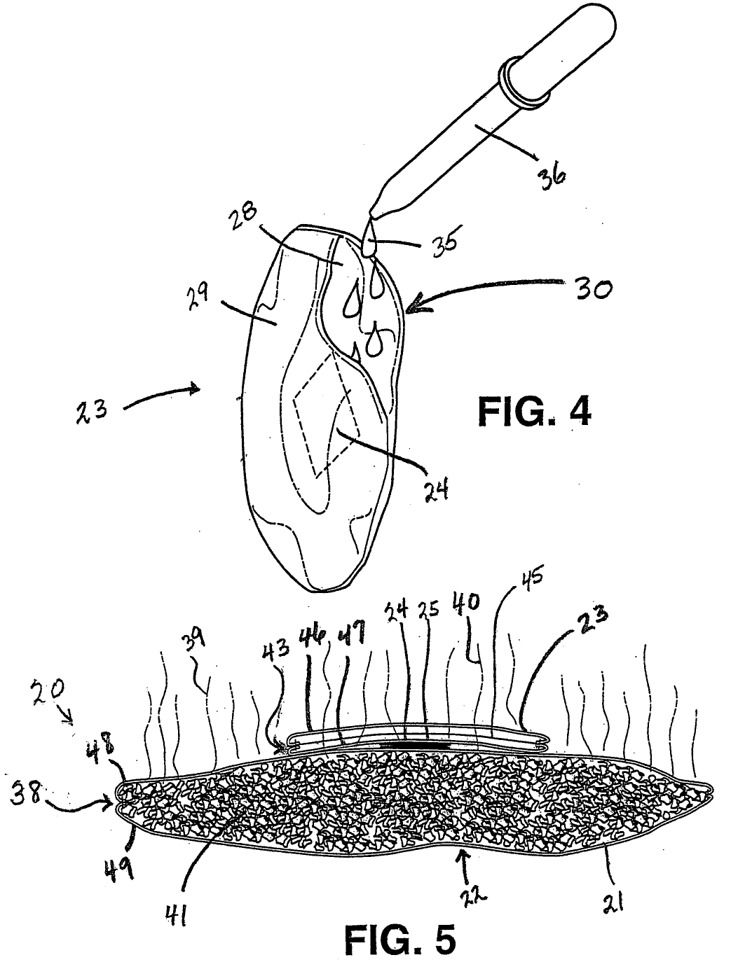

The vapor permeable pouch 23 comprises a first panel 46 attached to a second panel 47 along seam 43. The vapor permeable pouch 23 includes an outside surface 29 and an inside surface 28 (see FIG. 4).

The cushion 20 contains water-absorbing material 41 which, when heated, releases water vapor 39 through the top panel 48 and through the second panel 47 of the vapor permeable pouch 23. This causes vapor therapy materials 35 residing within absorption area 45 of the vapor permeable pouch 23 to become airborne as therapeutic vapor 40.

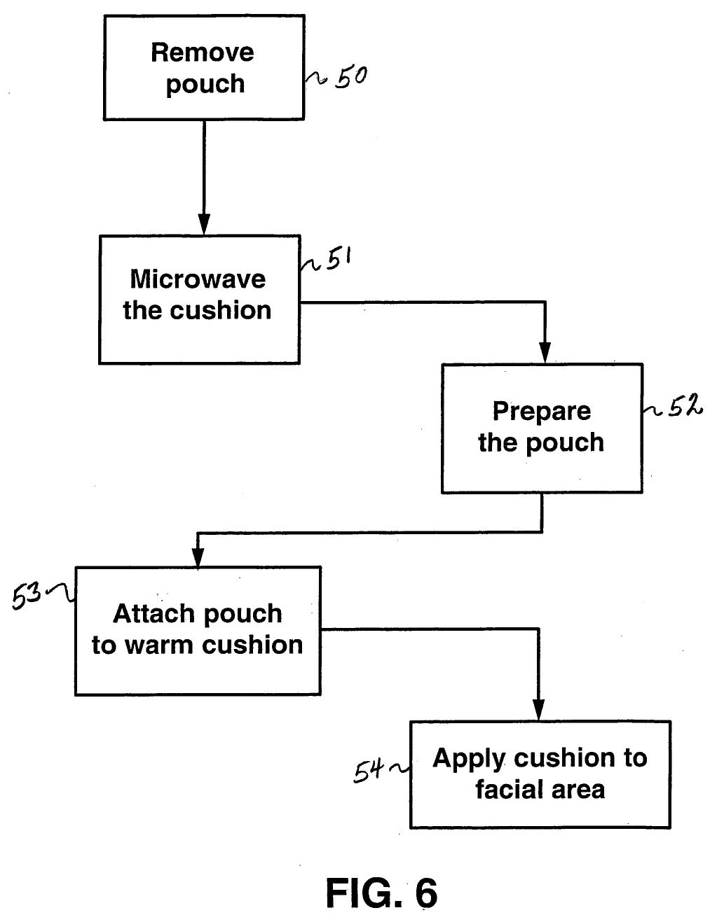

FIG. 6 is a schematic diagram showing one method of practicing the invention. Step 50 includes removing the vapor permeable pouch 23 from the cushion 20. Step 50 is unnecessary if the pouch 23 is already disengaged, as for example as shown in FIG. 2. It is usually undesirable to allow the vapor permeable pouch 23 to be subjected to microwave energy.

In one embodiment of the invention, in step 51, microwave energy is applied to the cushion 20 by placing the cushion 20 within a microwave oven 33 (FIG. 3). In other embodiments of the method, alternative sources of energy can be applied to the water absorbing material, as further discussed herein. However, a desirable and preferred method of generating water vapor release is to apply microwaves to the water absorbing material 41. The cushion 20 (without a pouch 23) is placed within the microwave oven 33, and the door 32 is closed. The cushion 20 is subjected to microwave heating for a period of time which varies between about 45 seconds and about 2 minutes, depending upon the amount of heat the user desires to apply in the particular application, and the power output of the microwave oven 33. Additional time variations may be practiced within the present subject matter, as determined by the user for their individual comfort level. Once the cushion 20 has been heated, the cushion 20 is removed from the microwave oven 33. Alternatively, the cushion 20 may be heated while in an automobile by applying the cushion 20 to car seats which have automatic heat control or have received a significant amount of sunlight upon their surface. In other applications, the cushion 20 may be heated by placing it near a source of radiant heat, such as a fireplace, stove, heater, or furnace. In some applications, the cushion 20 may be placed in a clothing dryer and circulated within the dryer to heat the cushion 20. In yet another embodiment of the invention, liquid water may be wiped upon the outer surface 22 of the vapor permeable cover 21 prior to heating the cushion 20 to supplement or boost water vapor release.

In a next step 52, the vapor permeable pouch 23 is prepared by applying vapor therapy materials 35 (also described herein as compositions) into or upon the vapor permeable pouch 23. In some embodiments of the invention, step 52 involves applying a vapor therapy material 35 which is a liquid upon an inside surface 28 of the vapor permeable pouch 23 (see FIG. 4). In the embodiment of the vapor permeable pouch 23 shown in FIG. 4, a dropper 36 is used to apply vapor therapy material 35 which is a liquid upon the inside surface 28 of the vapor permeable pouch 23. The vapor therapy material 35 may be absorbed upon the inside surface 28. Typically, the vapor therapy material 35 is applied to the vapor permeable pouch 23 while the pouch 23 is disengaged from the vapor permeable cover 21, as shown in FIG. 4.

Other applications of the invention may employ vapor therapy materials 35 which are solid, semi-solid, or flowable. In such applications, the materials 35 could be applied with a cotton swab or other wiping device upon outside surface 29 or inside surface 28 of the vapor permeable pouch 23. Some applications of the invention employ more than one variety of vapor therapy materials 35. That is, two or more types of vapor therapy materials 35 may be used at one time in the course of one treatment. Some applications of the invention may employ non-flowable solid particles such as potpourri or aromatherapy materials, for example.

In step 53, the vapor permeable pouch 23 which has been prepared according to step 52 is applied to the warmed cushion 20. This may occur after the cushion 20 has received microwave energy, thereby allowing the water absorbing material 41 within the cushion 20 to absorb heat.

Once the vapor permeable pouch 23 has been applied to the warm cushion 20 as in step 53, the cushion 20 may be applied to the facial area of a user as in step 54 of FIG. 6. Vapor therapy materials 35 are released from the vapor permeable pouch 23 with water vapor as gaseous therapeutic vapors 40 (see FIG. 5). When a person places the heated cushion 20 upon or near his or her face, the gaseous therapeutic vapor 40 becomes available for contact with the user. The user then may breathe the therapeutic vapor 40 through his or her mouth or nose. In other applications, the vapor 40 may provide benefits by opening pores and soothing the user's skin in the facial/neck area.

After a period of time, a user may renew the treatment by re-heating the cushion 20. This involves removing the pouch 23 from the cover 21, heating again the water absorbing material 41, and re-applying the pouch 23 to the cover 21. The pouch 23 also may receive once more vapor therapy materials 35 if needed or desired.

The therapeutic vapor 40 which contains heated water vapor may open up sinus cavities and supply needed respiratory relief to a user. In other applications of the invention, the cushion 20 may be applied to other body parts for pain relief or heat therapy.

The-vapor therapy treatment device of the invention may be used for relief of sinus pressure headaches, nasal itch, allergies, tension headaches, muscle pain, joint pain, backaches and the like. The cushion 20 is particularly useful in the relief of sinus headaches and related respiratory illnesses. The generation of heated water vapor without the necessity of adding liquid water to the device is a significant advantage in the practice of the invention. In one embodiment of the invention, the vapor permeable cover 21 is comprised of cotton flannel, which is particularly well adapted for skin comfort. The fiber of the flannel may be non-toxic and unscented. One flannel that is particularly useful in the application of the invention is “clothing grade” flannel, including for example middleweight flannel. Such flannel may be obtained from various U.S. manufacturers, including Marcus Brothers Fabric Company, Benatex Fabric Company, and Timeless Treasures Company. In one particular embodiment, a one-layer exterior flannel sheet is used. The flannel sheet may be seamed or stitched by commercial sewing machines in the manufacture of the cushion.

The water absorbing material 41 employed in the invention may include essentially any material capable of uptake of liquid or gaseous water, including material that adsorbs water from the air. Hygroscopic materials which seek moisture equilibrium with the atmosphere are particularly useful. Cellulose, for example, may be used as a water absorbing material 41 in the application of the invention. The exposure of cellulose to relative humidity causes cellulose to either swell or shrink, depending upon the relative humidity.

One material which has proved particularly useful in the application of the invention is a water absorbing material 41 is a cellulosic material manufactured and marketed by the Pig Corporation known as PIG® Lite-Dri® Absorbent. This absorbent is available in eight-gallon bags and is light gray in color. This product (item number PLP201) is distributed by the PIG Corporation, I Pork Avenue, Tipton, Pa. 16684-0304 (online products catalog at http://newpig.com). This product is a non-selective loose absorbent cellulose which is said to be capable of absorbing and containing oils and other fluids. The Lite-Dri® product is comprised of recycled newsprint (cellulose). Furthermore, a small amount of mineral oil, typically less than about 1%, is provided in the product. The mineral oil fraction functions primarily to control dust. The mineral oil acts as a binding agent with the cellulose. The Lite-Dri® product typically is provided in granular or pellet form, and tends to be highly absorptive of various types of materials. The Lite-Dri product absorbs water vapor from the air at ambient temperatures.

Various vapor therapy materials 35 may be applied in the practice of the invention. In some applications, the materials 35 include inhalation agents such as mentholated vapor products. In other applications, aromatherapy products and herbs, leaves, or floral potpourri may be used. In some applications, the vapor therapy compositions are scented, while in other applications they are unscented.

Various “over-the-counter” mentholatum products such as Vicks® Vapo-Rub®, for example, may be quite useful as vapor therapy materials for application in the invention. Asthma or headache relief agents may be employed as well, including both “over-the-counter” and prescription vapor therapy compositions, each of which should be used only under the supervision of a physician. Essentially any composition that is effectively absorbed in the lining of the nasal passages, or is soothing when applied to the face or skin in combination with warm water vapor, can be employed in the practice of the invention.

The cushion 20 may be provided in a variety of sizes or shapes. For example, the cushion 20 may be square, rectangular, triangular, or octagonal. It may be configured in fancy or whimsical shapes. In one useful embodiment, the cushion 20 is round in shape, and is about 11 inches in diameter. The cushion could range from about 5 inches up to as much as 15 inches or greater in diameter or length.

The amount of water absorbing material 41 within the inner space of the vapor permeable cover is typically between about 0.5 lbs. and 1 lb. In one preferred embodiment of the invention, the amount of water absorbing material used is 0.75 lbs. of Pig® Lite-Dri® absorbent material.

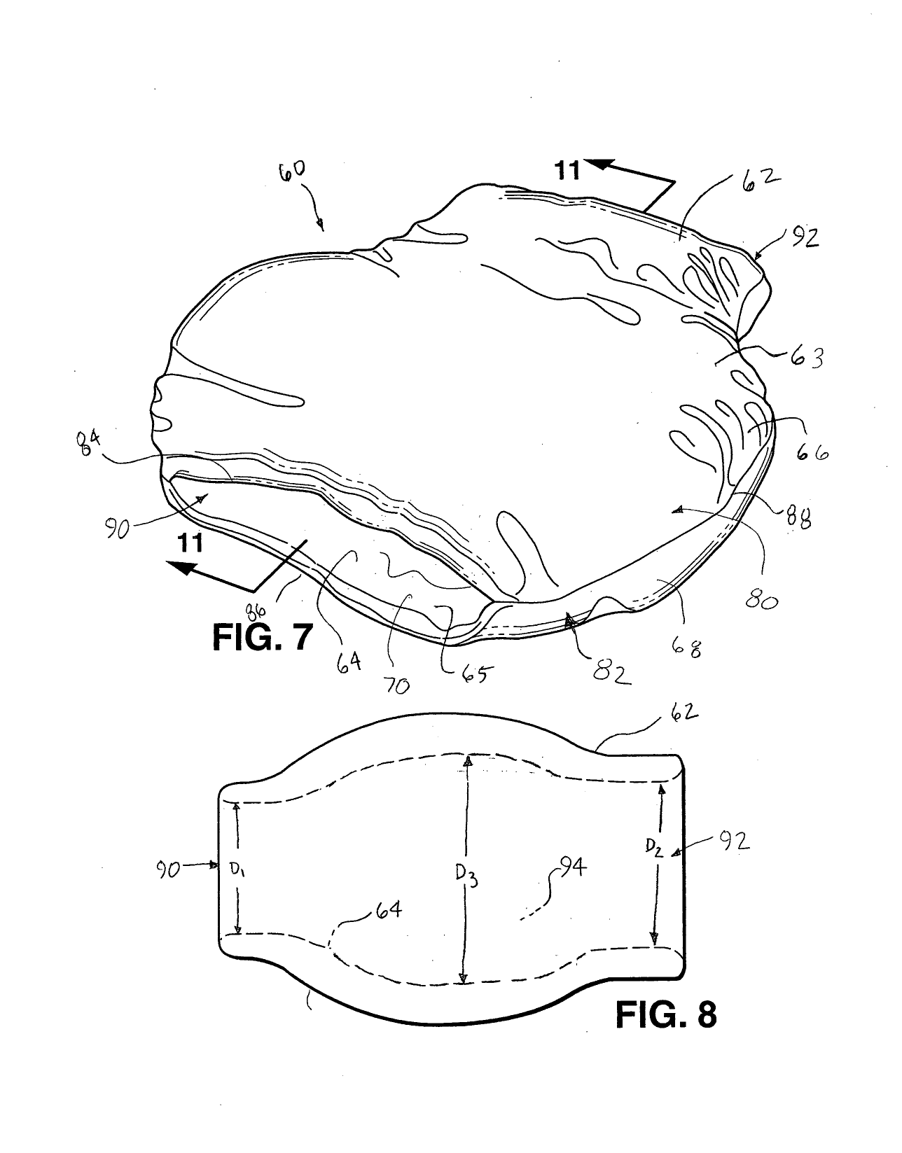

FIG. 7-11 shows an exemplary embodiment of a therapeutic device, generally 60, for use in obtaining relief from pains and aches caused by circulatory, neurological, muscle and joint ailments. The therapeutic device 60 is especially beneficial for use on appendages of a person who suffers from such ailments. The user may insert an appendage into a preheated therapeutic device 60 that is emanating a moist heat to receive relief from pain of such ailments. “Therapeutic” as used herein means to generally give relief from pain, aches and other ailments and does not have a specific medical connation related to medically prescribed treatments.

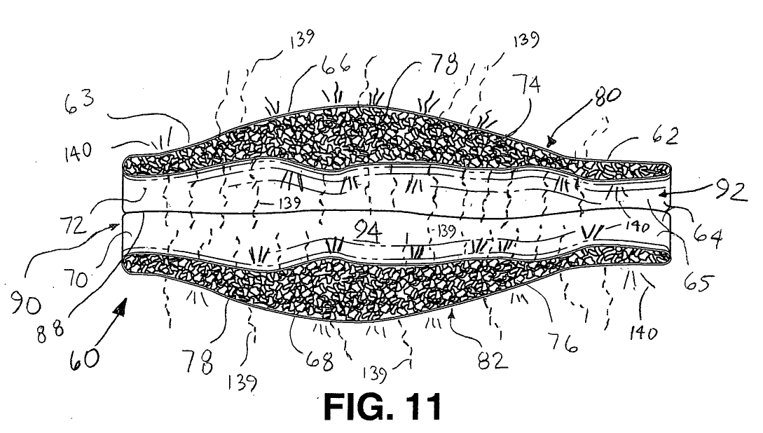

The therapeutic device 60 includes a vapor permeable outer cover 62 that is secured to a vapor permeable inner cover 64. These vapor permeable covers 62, 63 may be constructed on any vapor permeable film or fabric. In the embodiment shown, a vapor permeable outer cover 62 and the vapor permeable inner cover 64 comprise cotton flannel as described above with the vapor treatment device. As seen in the cross-sectional view of FIG. 11 taken along the lines 11 in FIG. 7, the vapor permeable outer cover 62 forms an outer surface 63 on the outside of the therapeutic device, while the vapor permeable inner cover 64 forms an inner surface 65 on a collapsible appendage receiving chamber 94 of the device 60 into which the appendage of the user may be inserted. In the shown embodiment, two opposing openings 90 and 92 are defined by the therapeutic device that lead into the chamber 94 to permit the insertion of such an appendage. In some embodiments, only one such opening may be defined. In other embodiments, multiple openings may be provided.

The vapor permeable outer cover 62 and the vapor permeable inner cover 64 define at least one enclosed interior space therebetween. In the shown embodiment, a first interior space 74 and a second interior space 76 is defined between the vapor permeable outer cover 62 and the vapor permeable inner cover 64. A water absorbent material 78 is disposed within the interior spaces 74, 76 defined between the vapor permeable outer cover 62 and the vapor permeable inner cover 64. Upon application of energy, this water absorbent material 78 is capable of water vapor release, so that the water absorbent material 78 is capable of releasing the water vapor through the vapor permeable outer cover 62 and the vapor permeable inner cover 64.

As described above with the vapor therapy device, the water absorbent material 78 may be capable of water uptake at ambient temperatures. The water absorbent material 78 may include essentially any material capable of uptake of liquid or gaseous water, including material that adsorbs water from the air. Hygroscopic materials which seek moisture equilibrium with the atmosphere are particularly useful. A water absorbent material that includes a cellulose containing material may be used. Pig® Lite-Dri® absorbent material, for example, can be used as the water absorbent material 78. The amount of water absorbent material may vary from about 1 lb. to 3 lbs. depending on the size of the therapeutic device 60.

To form the vapor permeable covers 62, 64, respectively, a first cushion 80 may attached to a second cushion 82 along a seam 88. The first cushion 80 may be attached to the second cushion 82 through any conventional means that is microwavable safe to form the seam 88. For instance, the first cushion 80 may be sewn to the second cushion 82. Further, the first cushion 80 may be attached to the second cushion 82 through the use of adhesives or the like or may be releasably attached through the use of snaps, hook and loop fasteners, or the like. The attachment of the first cushion 80 to the second cushion 82 allows the chamber 94 to be defined between the first and second cushions 80, 82 and also form at least one opening 90, 92 leading into the chamber 94. In the embodiment shown with two opposing openings 90, 92, the seam 88 is formed on either side of the openings 90, 92.

The first cushion 80 defines the first interior space 74 and the second cushion 82 defines the second interior space 76 with the water absorbent material 78 residing in both interior spaces 74, 76. The first cushion 80 can be made of a first outer panel 66 and a first inner panel 72 of material, such as a textile fabric. The second cushion 82 can be made of a second outer panel 68 and a second inner panel 70 of the same or similar material. For each cushion 80, 82, the outer panel 66, 68 may be sewn, or otherwise attached, to the respective inner panel 72, 70 to form the respective interior space 74, 76 so as to prevent any leakage of the water absorbent material 78 from therein. When a cushion 80, 82 is made from two panels 66, 72, 68, 70, the panels are attached along a seam 84, 86, respectively. In such embodiments, it can be beneficial for the sides of the two cushions 80, 82 along the seams 84, 86 to be joined along the seam 88. This allows the therapeutic device to lay flat.

FIG. 11 depicts the therapeutic device 60 after it has been heated. Upon applying energy to the therapeutic device 60 such as microwaves through the use of a microwave oven, the therapeutic device 60 including the water absorbent material 78 is heated. This heating of the water absorbent material 78 cause the release of water in the form of water vapor 139 shown in FIG. 11. The vapors 139 penetrate both the inner and outer covers 62, 64, respectively. Further, heat 140 radiates from the outer and inner covers 62, 64 and from the water absorbent material 78. In this manner, a moist heat emanates through the inner surface 65 to within the chamber 94 and from the outer surface 63 of the therapeutic device 60 into the surrounding environment. Within the chamber 94, the heat 140 and vapor 139 create a steamy environment into which an appendage may be placed. As with the vapor therapy device, however, the therapeutic device 60 may be heated in different manners as described above.

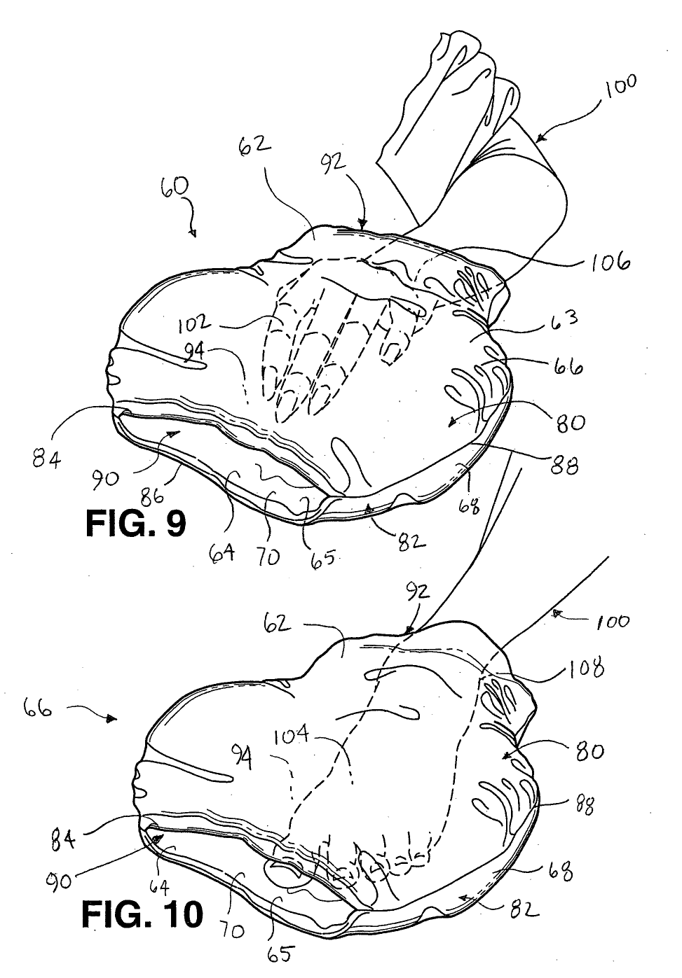

As seen in FIGS. 9 and 10, once the therapeutic device 60 is heated and the heat and heated water vapor is emitted therefrom, a user 85 may insert an appendage, such as a hand 102 or foot 104, through one of the openings 90, 92 into the chamber 94. The moist heat from the radiated heat 140 and the released vapor 139 surrounds the hand 102 or foot 104 to help relief the aches and pains caused by the ailment afflicting those appendages. The moist heat opens pores within the skin and improves the circulation within the appendage, for example, to help create such relief.

The therapeutic device 60 effectively surrounds the appendage, for example, the hand 102 or foot 104, and is molded around the respective appendage 102,104 due to the flexibility and draping quality of the outer and inner vapor permeable covers 62, 64 and the formation and/or composition of the water absorbent material 78. The chamber 94 is large enough to insert a large appendage. However, the draping quality of the inner vapor permeable cover 64 and the outer vapor permeable cover 62 and the composition of the water absorbent material 78 allow the chamber 94 to collapse around appendages that do not require the use of the entire space. This collapsibility of the chamber 94 increases efficiency and potency of the heating relief received by the appendage inserted therein.

For example, a textile fabric used as inner vapor permeable cover 64 can bend and turn (i.e., the drape) in a manner that allows the fabric to mold to the shape of the object on which it rests. Further, the water absorbent material 78 can comprise a conglomerate of pellets, fibers, strands of yarns, or other small objects, that individually may be rigid, but the water absorbent material 78 taken as a whole conglomerate will also mold to the shape of the object against which it rests. Thereby, the size of the chamber 94 is effectively adjustable to the size of the appendage which is inserted therein. This collapsible quality of the therapeutic device allows the volume of the chamber to be an appropriate size for the appendage to maximize the effectiveness of the heat 140 and vapors 139. The heat 140 and vapors 139 do not heat unneeded space within the chamber 94 thereby conserving to a certain extent the energy used to provide relief to the appendage and more efficiently using the heat 140 and vapors 139 released into the chamber 94.

The therapeutic device 60 may also be used on other areas of the body of the user 100 other than appendages which can be inserted into the chamber 94 of the device 60. The outer cover 62 of the therapeutic device 60 can be used as a compress once the device is heated. For example, once heated, the device 60 may be pressed again the back of the head or neck of the user 100 to remedy headaches. The moist heat from the heat 140 and the vapor 139 emanating from the portion of the outer cover 62 can provide a moist hot compress to increase circulation of the blood in or to the afflicted area to help relief the pain felt by the user 100.

The top view of the therapeutic device in FIG. 8 shows the therapeutic device lying flat so that the first inner panel resides against the second inner panel. As can be seen from this view, width D₂ of opening 92 is wider than the width D₁ of opening 90. The wider opening 92 allows for the larger appendages to be inserted into the chamber 94. Further, the width D₃ of the chamber 94 is larger than either of the openings 90, 92. The widths D₁, D₂ of the openings 90, 92 when the therapeutic device 60 is lying flat allow the openings 90, 92 to expand to a cross-sectional area large enough to receive an appendage. The larger width D3 of the chamber 94 allows an appendage, such as a hand to spread out for receipt of more effective relief. The larger width D₂ of the opening 92 as compared to the width D₁ of opening 90 and the larger width D₃ of the chamber 94 as compared to the widths D₁, D₂ of the openings 90, 92 translate into larger cross-sections, respectively. In this manner the openings 90, 92 serve as an entryway which is smaller than the size of the appendage receiving chamber 94.

The cross-sectional size of the openings 90, 92 and the positioning of the openings at opposing ends of the chamber 94 can be designed to allow the therapeutic device 60 to be used on the elbow or upper arm of the user 100, for example. By one of the openings 90, 92 possessing a larger cross-sectional area, the larger opening 92 may be able to accept a larger appendage, while the smaller opening 90 may be used to receive a smaller appendage.

The arrangement of the vapor permeable covers to form the therapeutic device may take on different forms. For example, a single rectangular piece of vapor permeable cover material may be used to form the outer and inner surface of the therapeutic device where two opposing sides of the piece of material are secured together to form a continuous piece of material. One of the unattached sides may be turned outward so as to extent back toward the remaining unattached side of the material to create a fold for receipt of the water absorbent material. The unattached sides may then be secured together to form an outer cover that is secured to an inner cover. Similarly, a tube of a circular knit fabric can be used in a like manner.

Multiple outer panels secured to one another to form an outer cover and multiple inner panels secured to one another to form an inner cover can also be employed. The outer panels can be connected together to form a continuous piece of material with two unattached sides to comprise the outer cover, and the inner panels can be connected together to also form a continuous piece of material with two unattached sides to comprise the inner cover. The inner cover of continuous connected inner panels is inserted into the outer cover of continuous connected outer panels. An unattached side of the inner cover is secured to an unattached side of the outer cover that is aligned with that side of the inner cover. This creates a fold, or interior space, between the inner cover and outer cover for receipt of the water absorbent material. The other unattached side of the inner cover and the other unattached side of the outer cover are secured to enclose the interior space filled with water absorbent material. In this manner, an appendage receiving chamber with two openings is created by the inner cover. Further, a single interior space is created between the inner cover and outer cover.

A method for producing therapeutic relief in an appendage of a user using a therapeutic device is further described below. To start, a vapor permeable outer cover is secured to a vapor permeable inner cover defining an interior space between the outer cover and inner cover. The outer cover and the inner cover are configured so that the inner cover defines a collapsible appendage receiving chamber and an opening. Further, a water absorbent material is provided within the interior space between the outer cover and the inner cover.

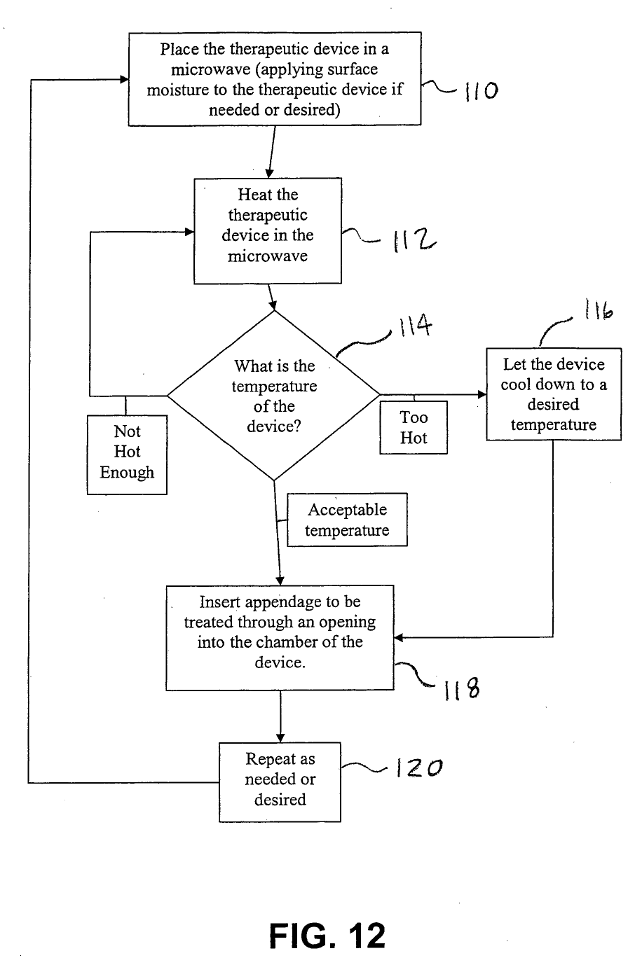

After the therapeutic device is provided, the outer cover, the inner cover and the water absorbent material are heated by applying energy thereto. This may be done, for example, through steps 110 and 112 shown in FIG. 12. As in step 110, the therapeutic device may be placed in a microwave oven as shown in FIG. 3. If it is desired, the user may apply a small amount of moisture to the outer surface of the outer cover of the therapeutic device. The moisture would pass through the outer cover, at least to a certain extent, to be absorbed by the water absorbent material. In this manner, a larger amount of heated water vapor will be produced creating a more intense amount of the moist heat to provide relief to the user's ailment. However, as stated above, the addition of extra moisture is not necessary to produce the moist heat from the water absorbent material.

In step 112, the therapeutic device is heated in the microwave. The vapor permeable covers are also permeable to the microwaves to allow the heating of the water absorbent material. The therapeutic device would preferably be heated on both sides of the therapeutic device. Depending on the power of the microwave, the amount of time needed to microwave the therapeutic device will vary. The therapeutic device can be subjected to microwave heating for a period of time which varies between about 1 minute and about 4 minutes, depending upon the amount of heat the user desires to apply in the particular application, and the power output of the microwave oven. For example, for a 600-800 watt microwave, the therapeutic device may be heated for a suggested time of about 70 seconds on one side. Once that side is heated, the microwave should be opened, the therapeutic device should be flipped, and the other side should be heated for the same time period. For an 800-1,000 watt microwave, the therapeutic device may be heated for a suggested period of about one minute per side. Further, for a 1,000-1,500 watt microwave, the therapeutic device may be heated for a suggested period of 40 seconds per side.

Once the therapeutic device is heated, the temperature of the therapeutic device should be check to see if the device is at a desirable temperature as outlined in step 114. If it is not, step 112 is repeated. However, the amount of time needed to heat the therapeutic device will be lessoned due to the fact that the device is already partially heated. For example, the therapeutic device can be heated on each side for ten or fifteen second intervals repeating steps 112 and 114 until a desired temperature is reached. If the temperature of the device is too hot in step 114, then, in step 116, the device should be allowed to cool down to the desired temperature.

If the temperature of the device is determined to be acceptable in step 114, or upon letting the device cool to a desired temperature in step 116, the afflicted appendage of the user is inserted through the opening and into the chamber, which are defined by the inner cover, as outlined in step 118. Heat is radiated and water vapor is released from the water absorbent material to treat the appendage disposed within the chamber of the inner cover. Heat may also radiate from the outer cover and the inner cover. If sufficient relief is realized by the user, he/she can remove the appendage and go about his/her business. If the user feels he/she has not received adequate relief or if the user has another appendage to be treated upon the therapeutic device cooling down, the method can be repeated as needed or desired in step 120.

It is understood by one of ordinary skill in the art that the present discussion is a description of exemplary embodiments only, and is not intended as limiting the broader aspects of the present invention, which broader aspects are embodied in the exemplary constructions. The invention is shown by example in the appended claims.

1. A therapeutic device, said device comprising:
a vapor permeable outer cover secured to a vapor permeable inner cover, said vapor permeable outer cover and said vapor permeable inner cover defining at least one enclosed interior space between said vapor permeable outer cover and said vapor permeable inner cover with said vapor permeable outer cover and said vapor permeable inner cover being permeable to microwaves;
said vapor permeable outer cover defining an outer surface and said vapor permeable inner cover defining an inner surface with said inner surface of said vapor permeable inner cover defining a collapsible appendage receiving chamber and at least one opening to said appendage receiving chamber, said at least one opening and said appendage receiving chamber capable of receiving an appendage of a user with said appendage receiving chamber being larger in cross-sectional size than said at least one opening;
water absorbent material disposed within said at least one interior space defined between said vapor permeable outer cover and said vapor permeable inner cover, said water absorbent material being capable of water vapor release upon application of energy to said water absorbent material, so that said water absorbent material is capable of releasing said water vapor through said vapor permeable outer cover and said vapor permeable inner cover.

2. A device as in claim 1, wherein said water absorbent material is capable of water uptake at ambient temperatures.

3. A device as in claim 1, wherein said water absorbent material comprises cellulose-containing absorbent material.

4. A device as in claim 1, wherein said at least one opening comprises two opposing openings at either end of said collapsible appendage receiving chamber.

5. A device as in claim 4, wherein one opening of said two openings is larger in cross-sectional size than the other opening.

6. A device as in claim 1, wherein said vapor permeable outer cover comprises a first outer panel forming a first portion of said outer surface and a second outer panel forming a second portion of said outer surface and said vapor permeable inner cover comprises a first inner panel forming a first portion of said inner surface and a second inner panel forming a second portion of said inner surface.

7. A device as in claim 6, wherein said at least one enclosed interior space comprises a first interior space and a second interior space with said first outer panel and said first inner panel defining said first interior space and said second outer panel and said second inner panel defining said second interior space.

8. A device as in claim 1, wherein said water absorbent material radiates heat through said vapor permeable outer cover and said vapor permeable inner cover.

9. A device as in claim 1, wherein said inner vapor permeable cover drapes around the appendage of the user when the appendage is disposed in said collapsible appendage receiving chamber so as to effective surround the appendage.

10. A device as in claim 1, wherein said vapor permeable outer cover and said vapor permeable inner cover comprises a textile fabric.

11. A method for producing therapeutic relief in an appendage of a user of a therapeutic device, said method comprising:
providing a vapor permeable outer cover secured to a vapor permeable inner cover defining an interior space between the outer cover and inner cover and configured so that the inner cover defines a collapsible appendage receiving chamber and an opening with the appendage receiving chamber being larger in cross-sectional size than the opening;
providing water absorbent material within the interior space between the outer cover and the inner cover;
heating the water absorbent material by applying energy thereto;
inserting an appendage of the user into the collapsible chamber through the opening defined by the inner cover; and
radiating heat and releasing water vapor from the water absorbent material to provide relief to the appendage disposed within the chamber of the inner cover.

12. A method as in claim 11, wherein the heating step includes heating the outer and inner covers by applying energy thereto, and the radiating step includes heat radiating from the outer cover and the inner cover.

13. A method as in claim 11, wherein the heating step includes the application of microwaves to the outer cover, inner cover, and water absorbent material.

14. A method as in claim 11, further comprising applying water to at least one of said outer cover or said inner cover prior to the heat step.

15. A therapeutic device, said device comprising:
a first cushion having a first vapor permeable cover defining an enclosed first interior space;
a second cushion having a second vapor permeable cover defining an enclosed second interior space;
said first cushion secured to said second cushion so that a collapsible chamber is defined between said first and second cushions, said first and second cushions forming at least one opening leading into said collapsible chamber;
water absorbent material positioned within said first interior space and said second interior space, said water absorbent material being capable of water uptake at ambient temperatures and water vapor release upon application of energy to said water absorbent material; and
said first and second cushions draping around an appendage of a user when the appendage is disposed in said collapsible chamber so as to effective surround the appendage.

16. A device as in claim 15, wherein said first and second vapor permeable covers are permeable to microwaves.

17. A device as in claim 16, wherein said first and second vapor permeable covers and said water absorbent material radiate heat upon application of microwaves.

18. A device as in claim 15, wherein said at least one opening comprises two opposing openings at either end of said collapsible chamber.

19. A device as in claim 18, wherein one opening of said two openings is larger in cross-sectional size than the other opening.

20. A therapeutic device, said device comprising:
a first textile outer cover sewn to a first textile inner cover to form a surface of a first cushion, said first textile outer cover and said first textile inner cover defining a first enclosed interior space of said first cushion;
a second textile outer cover sewn to a second textile inner cover to form a surface of a second cushion, said second textile outer cover and said second textile inner cover defining a second enclosed interior space of said second cushion;
said first textile outer and inner covers sewn to said second textile outer and inner covers so as to define a collapsible appendage receiving chamber having two opposing openings between said first textile inner cover of said first cushion and said second textile inner cover of said second cushion;
one of said two opposing openings being larger in cross-section than the other of said two opposing openings and said collapsible appendage receiving chamber being larger in cross-section than both of said two opposing openings; and
water absorbent material disposed within said first interior space of said first cushion and within said second interior space of said second cushion, said water absorbent material being capable of water vapor release upon application of energy to said water absorbent material, so that said water absorbent material is capable of releasing said water vapor through said first and second textile outer covers and said first and second inner textile covers.
